(19) United States
(12) Patent Application Publication
(10) Pub. No.: US 2025/0186851 A1
(43) Pub. Date: Jun. 12, 2025

(54) EXERCISE SYSTEM AND METHOD OF ITS USE

(71) Applicant: Robert Shafie, Sharon, MA (US)

(72) Inventor: Robert Shafie, Sharon, MA (US)

(21) Appl. No.: 18/530,317

(22) Filed: Dec. 6, 2023

(51) Int. Cl. A63B 69/00; A61M 21/02

(52) U.S. Cl. CPC A63B 69/0086; A61M 21/02; A63B 69/002; A61M 2205/056; A63B 2209/10; A63B 2214/00; A63B 2243/0025

(57) ABSTRACT

The present invention provides an exercise system, which aims to integrate physical activity with mental engagement, providing a tool for individuals, including athletes and those facing mental or physical impairments, to enhance their overall health. By attaching a ball to the user's body during various activities, this system promotes both physical exercise and mental involvement, potentially reducing stress, anxiety, and enhancing cognitive performance. Moreover, this system has the potential to assist in managing various health conditions and improving overall well-being, making it a versatile solution for a wide range of users.

### TECHNICAL FIELD

Embodiments disclosed herein relate generally to an exercise system and more specifically to an exercise system incorporating a ball and related method of its use.

### BACKGROUND OF THE INVENTION

Many individuals, if not the majority, either avoid physical exercise altogether or lack interest, engaging reluctantly in specific activities solely to maintain their physical and mental well-being. However, there is a universal appeal in playing with a ball across diverse backgrounds and values. The primary focus of this invention is an exercise system that secures a ball to the user's body during activities like walking, talking, standing, exercising, or showcasing talents. Additionally, it serves as an effective tool for physical therapy. This system aims to enhance overall body fitness, encompassing balance, coordination, and mental acuity through active meditation. Specifically beneficial for soccer players, it facilitates gradual physical and mental preparation before entering the game. Designed for individuals from ages five to ninety, regardless of gender, this system not only encourages exercise but also offers psychological benefits. Engaging in physical movement can alleviate symptoms associated with conditions like borderline personality disorder (BPD), depression, anxiety, and other mental health disorders, functioning as a form of behavioral therapy.

According to the Anxiety and Depression Association of America, just five minutes of aerobic exercise can trigger anti-anxiety effects. Utilizing the exercise system outlined in this invention for warm-up purposes elevates the user's heart rate, consequently enhancing blood flow. This heightened circulation allows increased oxygen delivery to the muscles, fostering improved muscular performance. The significance of physical fitness cannot be overstated, with its five key components being cardiovascular endurance, muscular strength, muscular endurance, flexibility, and body composition. Engaging in physical activity offers numerous benefits, including better brain health, weight management, reduced disease risk, strengthened bones and muscles, and improved capability to perform daily tasks. Adults who reduce sedentary behavior and engage in moderate-to-vigorous physical activity experience various health advantages. These include decreased risk of heart disease, better blood sugar and insulin level management, support in smoking cessation, enhanced mental health, sustained cognitive abilities with age, and weight control. The exercise system outlined in this invention is seen as a measure of the body's capacity to function efficiently and effectively in work, leisure, and emergency situations, as well as to pursue recreational activities without risk of injury.

The exercise system introduced in this invention aims to enhance mental engagement and preparedness, addressing issues such as anxiety, stress, excessive screen time (texting, internet use), and conditions like OCD. Moreover, it shows potential in ameliorating symptoms in individuals displaying early signs of Parkinson's disease, Huntington's disease, schizophrenia, and various mental and physical impairments. These potential improvements include reduced stress hormone levels, enhanced sleep quality, strengthened social connections, promotion of positive health behaviors, diminished suicidal thoughts, improved stress management, alleviation of chronic pain, mitigation of social isolation, and bolstering of self-esteem. Engaging in exercise triggers a mild stimulation to the brain's reward centers: the system that governs anticipation of pleasure, motivation, and hope. With consistent exercise, this system undergoes remodeling, resulting in heightened dopamine levels circulating in the body and an increase in available dopamine receptors over time.

It's typical for athletes to experience slight nervousness before a game or competition, as it primes the body for action by increasing stress hormone levels. Yet, when anxiety becomes overwhelming, it can significantly impact athletic performance. Soccer players, particularly substitutes on the sideline aiming to warm up, can significantly benefit from the exercise system outlined in this invention. It serves to alleviate pressure and anxiety before stepping onto the field, contributing to improved readiness for the game. By utilizing this exercise system, substitutes on the sideline not only sense the ball's physical presence but also become mentally and physically engaged, fostering their commitment and preparedness for the upcoming game.

Regular engagement in daily exercises through the system outlined in this invention enables users to enhance both their physical and mental well-being. This routine diminishes the risk of developing various diseases such as obesity, type 2 diabetes, and high blood pressure. Additionally, it aids in maintaining a healthy weight and potentially enhances an individual's cognitive performance, contributing to improved psychological well-being.

The development of an exercise system is crucial to address these challenges. This system aims to integrate physical activity with mental engagement, providing a tool for individuals, including athletes and those facing mental or physical impairments, to enhance their overall health. By attaching a ball to the user's body during various activities, this system promotes both physical exercise and mental involvement, potentially reducing stress, anxiety, and enhancing cognitive performance. Moreover, this system has the potential to assist in managing various health conditions and improving overall well-being, making it a versatile solution for a wide range of users.

There's a clear need for an integrated exercise system that not only facilitates physical activity but also targets mental involvement and readiness. This system is essential for athletes seeking to optimize their performance by managing pre-game anxiety. Moreover, it serves as a solution for individuals dealing with a spectrum of health challenges, from obesity and diabetes to anxiety disorders and neurodegenerative diseases. By combining physical exercise with mental engagement, this system fills the gap in promoting holistic well-being and offers a versatile approach to improving health across various demographics and conditions.

### SUMMARY OF THE EMBODIMENTS

The present invention provides an exercise system for integrating physical activity with mental engagement to promote both physical exercise and mental involvement, reducing stress, anxiety, and enhancing cognitive performance as well as improving overall well-being. The system includes a harness adapted to wrap around and hold a ball coupled to a length adjustable belt by a length adjustable band.

In some instances, the corners of the harness each terminates in a stripe for wrapping around and holding the ball. The harness can further include two crossed extension stripes attached to one of the stripes of the harness. According to some embodiments of the present invention, the harness is a net adapted to receive and hold the ball. In some instances, the harness is formed by a plurality of longitudinally oriented bands supportingly attached to a plurality of laterally oriented bands at cross points, wherein the bands are substantially thin smooth surfaced bands to minimize radial protrusion from the surface of the ball.

The band can be permanently attached to the belt and the harness by adhesive, sewing, staples, or other suitable attaching means. In some instances, the band can be releasably attached to the belt and the harness using such attaching means as Velcro strips, D-rings, carabiners, hook and loops, or their combination. The band and/or belt can be elastic. According to some embodiments of the present invention, the system can further include a pouch for receiving and storing one or more items, such as folded exercise system, wallet, credit card and the like.

The method of using the exercise system is also disclosed. The method includes the steps of providing the exercise system comprising a harness adapted to wrap around and hold a ball coupled to a length adjustable belt by a length adjustable band;
encircling the harness around the ball; securing the belt around the user's waist; adjusting the length of the band as to position the ball at or slightly above the knee level; and kicking the ball using either knee.

Other aspects, embodiments and features of the undershirt and method will become apparent from the following detailed description when considered in conjunction with the accompanying figures. The accompanying figures are for schematic purposes and are not intended to be drawn to scale. In the figures, each identical or substantially similar component that is illustrated in various figures is represented by a single numeral or notation. For purposes of clarity, not every component is labeled in every figure. Nor is every component of each embodiment of the device and method shown where illustration is not necessary to allow those of ordinary skill in the art to understand the device and method.

### BRIEF DESCRIPTION OF THE DRAWINGS

The preceding summary, as well as the following detailed description of the disclosed exercise system and method, will be better understood when read in conjunction with the attached drawings. It should be understood, however, that neither the device nor the method is limited to the precise arrangements and instrumentalities shown.

FIG. 1 is a front perspective view of the exercise system of the present invention.

FIG. 2 is a back perspective view of the exercise system of the present invention.

FIG. 3 is a top view of the harness of the exercise system of the present invention.

FIG. 4 is a bottom view of the harness of the exercise system of the present invention.

FIG. 5 is a top view of the harness straps of the exercise system of the present invention.

FIG. 6 is a front perspective view of the system incorporating a soccer ball, in accordance with embodiments of the present invention.

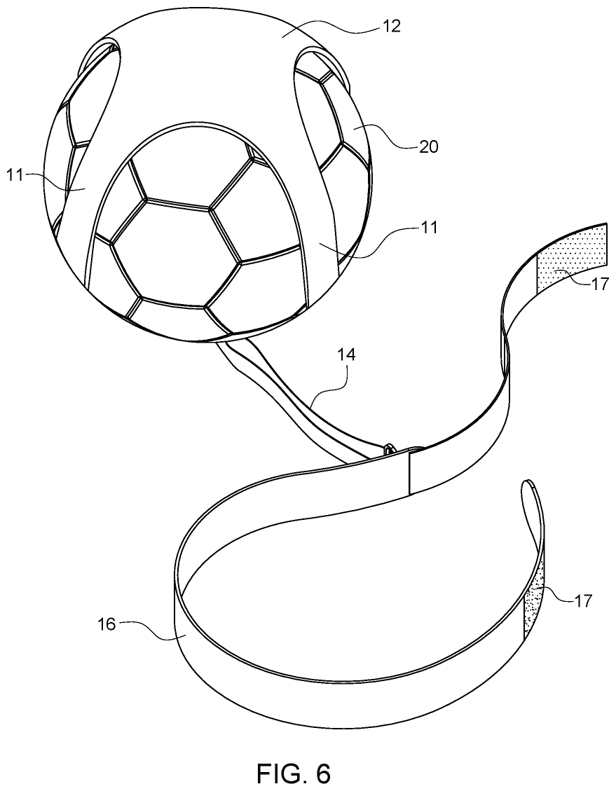

FIG. 7 is a side perspective view of the system incorporating a soccer ball, in accordance with embodiments of the present invention.

FIG. 8 is a perspective view of the exercise system in use, in accordance with a method of the present invention.

FIG. 9 is a front view of the pouch attachable to the exercise system of the present invention.

FIG. 10 is a back view of the pouch shown in FIG. 9.

### DETAILED DESCRIPTION OF SPECIFIC EMBODIMENTS

The present invention provides an exercise system, which aims to integrate physical activity with mental engagement, providing a tool for individuals, including athletes and those facing mental or physical impairments, to enhance their overall health. By attaching a ball to the user's body during various activities, this system promotes both physical exercise and mental involvement, potentially reducing stress, anxiety, and enhancing cognitive performance. Moreover, this system has the potential to assist in managing various health conditions and improving overall well-being, making it a versatile solution for a wide range of users.

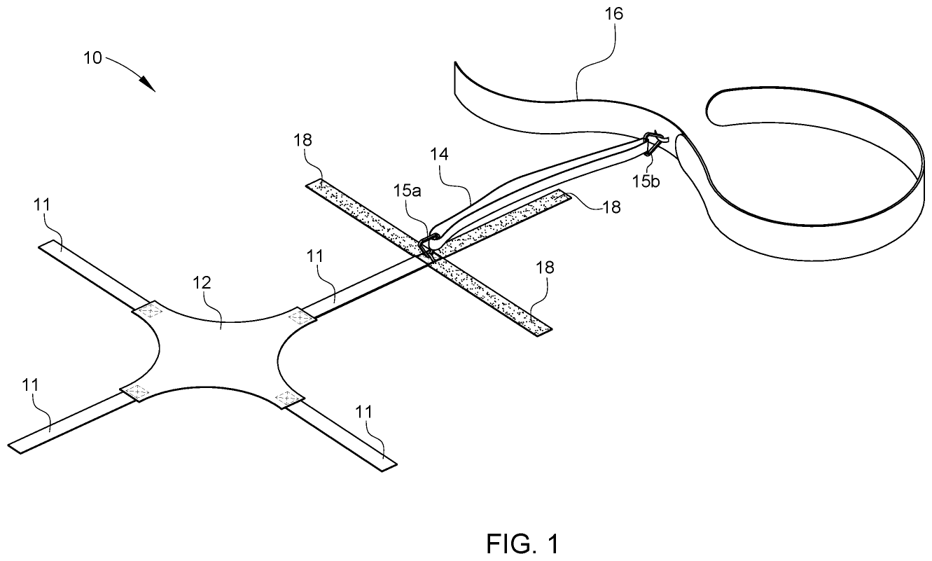

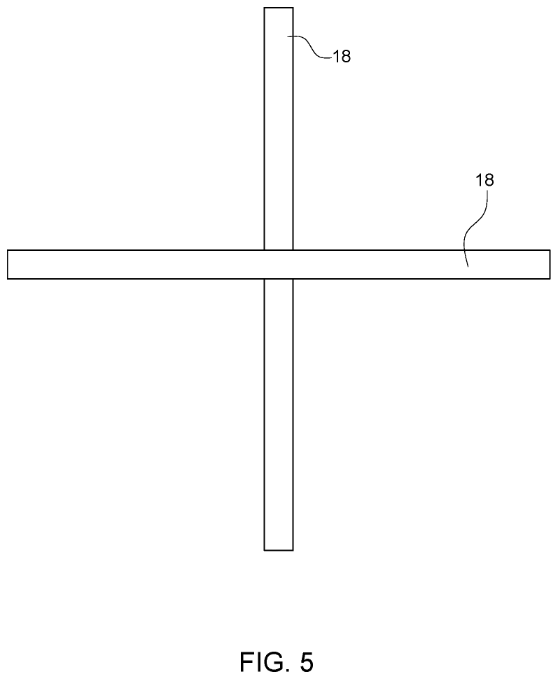

As illustrated in FIG. 1, the exercise system 10 includes harness 12 having four stripes 11 attached to the corners of harness 12 for wrapping around and holding a ball coupled to adjustable belt 16 by band 14, which can be permanently attached to belt 16 and harness 12. In some instances, band 14 can be permanently or releasably attached to harness 12 by ring 15a and to belt 16 by ring 15b. Rings 15a and 15b can be D-rings or carabiners or any other suitable rings such as O-rings, for example. Harness 12 can further include two crossed extension stripes 18 as shown in FIG. 5, for example. The harness is adapted to receive a ball of various types and sizes, such as a soccer ball, volleyball ball, basketball ball, or any other suitable ball.

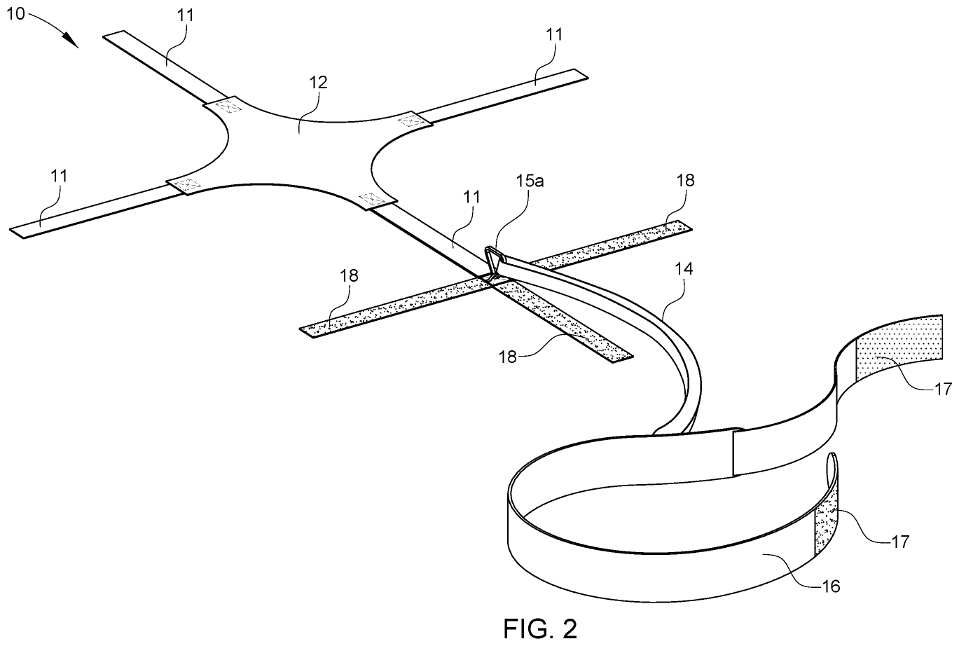

The permanent attachment can be realized by using glue or sewing with thread, or other similar means. The releasable attachment means can be Velcro, hook and loops, or any other suitable attached means. The belt 16 is designed to be worn around the waist of a user and the length of the belt 16 can be adjusted with Velcro strips 17 on both ends of the belt material as shown in FIG. 2, for example. It will be understood by a person skilled in the art that in addition to Velcro strips (hook and loop type configuration) other length adjusting means can also be employed. For example, the belt can have a slide buckle, or a D-ring buckle configuration.

Alternatively, the belt can be an elastic band with buttonholes and buttons to adjust the belt by fastening the buttons into different buttonholes to achieve the desired length. The length of the band 14 can be also adjusted in a similar fashion using Velcro or other suitable length adjusting means as discussed above in relation to the belt. The band 14 can be elastic. The length of the band 14 can be adjusted employing various length adjusting means such as slide buckle (to lengthen the band by pulling the loose end of the band through the buckle to loosen it and to shorten the belt, pulling the loose end of the band away from the buckle to tighten it), or D-ring buckle (pulling the band through the D-ring tightens the band while loosening is achieved by releasing the strap), Velcro closure (lengthen by detaching the Velcro strips and reposition them to increase the overall length of the belt, ensuring the Velcro strips are securely fastened for proper closure; shorten by overlapping the Velcro strips less to decrease the overall length of the band and adjusting the overlap until the desired length is achieved). The length of the band 14 can vary from 5 inches to 15 inches. In some instances, the length of the band can vary from 6 inches to 12 inches. According to a preferred embodiment, the maximum length of the band 14 is 11 inches (27.94 cm). According to another embodiment of the present invention, the band 14 is not adjustable but has a predetermined fixed length (for example, the fixed length of the band can be equal to 10 inches, or 11 inches, 12 inches etc.). In a preferred embodiment, the length of the band 14 is equal to 11 inches.

Other length adjusting means can include elastic band with buttonholes (to lengthen the band by unbuttoning the elastic band from its current buttonhole and reattach it to a further buttonhole for a longer length, to shorten the band by buttoning the elastic band into a higher or closer buttonhole to reduce the length; or snap fasteners (to lengthen the band by unsnapping the fasteners and reattaching them at a different point along the belt to increase the length, to shorten, snapping the fasteners into a closer or higher position on the belt to decrease its length; a slide and lock mechanism (to lengthen the band by pulling the loose end of the belt through the slide mechanism to release and extend the length, locking the band at the desired length by securing it with the slide-and-lock mechanism, and to shorten the band by pushing the slide mechanism to unlock and then pulling the loose end to shorten the belt, locking it again at the desired length); or drawstring closure (to lengthen the band by loosening the cord stopper and pulling the cord ends to increase the length, tightening the cord stopper once the desired length is achieved, to shorten the band by loosening the cord stopper and releasing some of the cord length to decrease the belt size, tightening the cord stopper to secure the adjusted length).

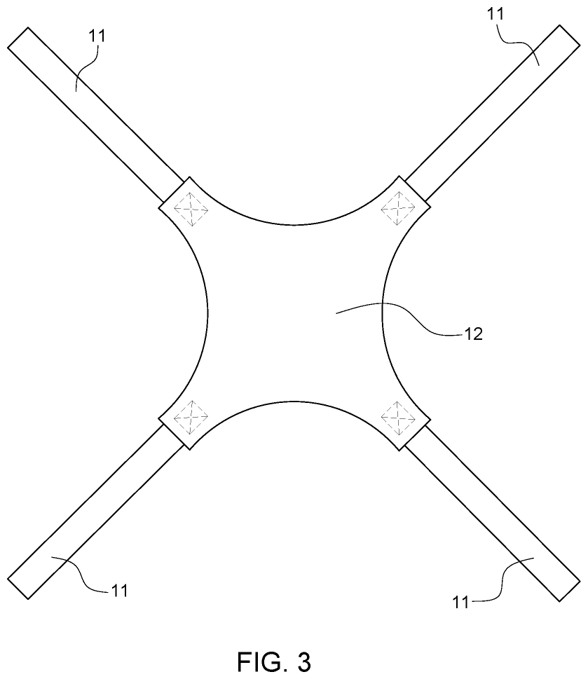

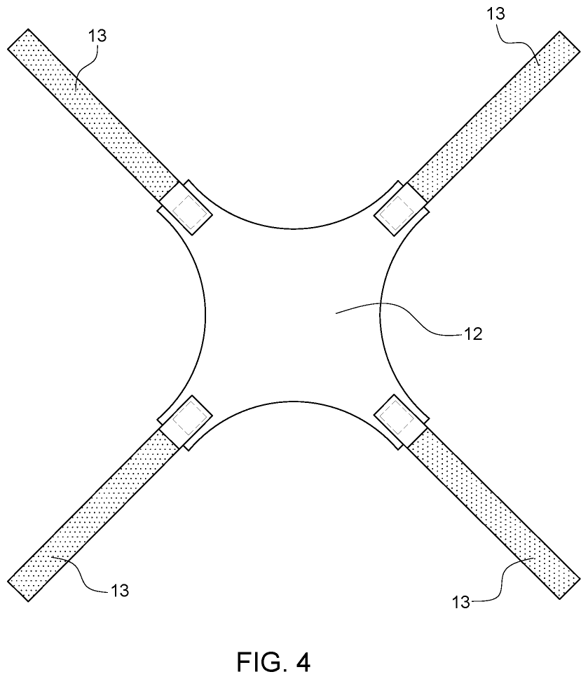

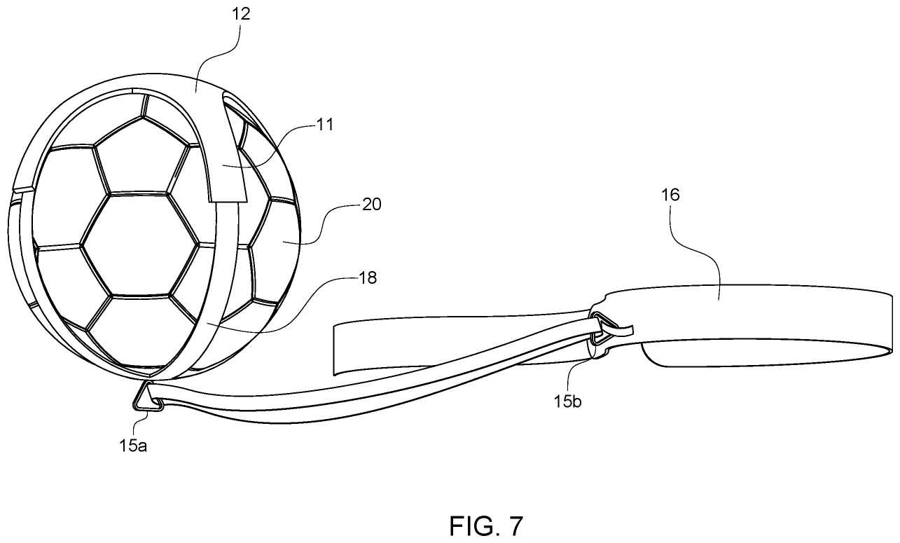

As shown in FIG. 3, harness 12 includes four stripes 11 attached at each corner of the harness. The bottom of each stripe 11 further include Velcro material 13 as illustrated in FIG. 4, for attachment to two crossed extension stripes 18, as further illustrated in FIG. 7. According to another embodiment of the present invention, harness 12 can be a net designed to receive and hold a ball such as a soccer ball or volleyball or any other suitable ball. In some instances, the harness can be formed by a plurality of longitudinally oriented bands supportingly attached to a plurality of laterally oriented bands at cross points. The bands should be relatively thin smooth surfaced bands to minimize radial protrusion from the surface of a ball.

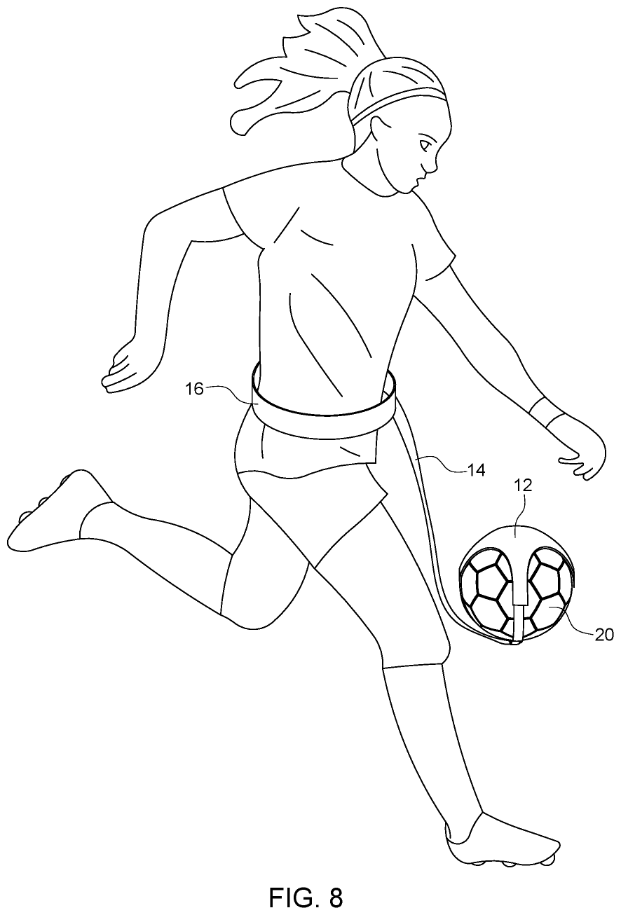

As shown in FIG. 8, the procedure for utilizing the exercise system of the current invention involves the encircling of harness 12 around ball 20. Subsequently, belt 16 is secured around the user's waist, and the length of band 14 is adjusted to position ball 20 at or slightly above knee level. Following this setup, the user engages in kicking the ball using either knee. The adjustable band length in this soccer ball harness invention is fundamental for precision and versatility in training. By customizing the band's length, the ball's position must be specifically set at or slightly above knee level. This deliberate positioning allows users to practice and refine their kicking techniques with both knees, fostering a comprehensive skill set. The ability to target knee-level positioning with the adjustable band elevates the training experience, emphasizing diverse kicking techniques and promoting a more comprehensive skill development in soccer players.

In addition to refining soccer skills, the ability to position the ball precisely at or slightly above the knee level through the adjustable band holds significant therapeutic value. For individuals undergoing physical therapy or those with impaired mobility, manipulating the ball at knee level offers a more accessible and controlled environment for rehabilitation or skill development. This feature caters to varying abilities, providing an inclusive platform where individuals with limited mobility can comfortably engage in ball manipulation exercises. By enabling manipulation at knee level, the invention extends its utility beyond regular soccer training, fostering inclusivity and facilitating rehabilitation for individuals with different physical abilities.

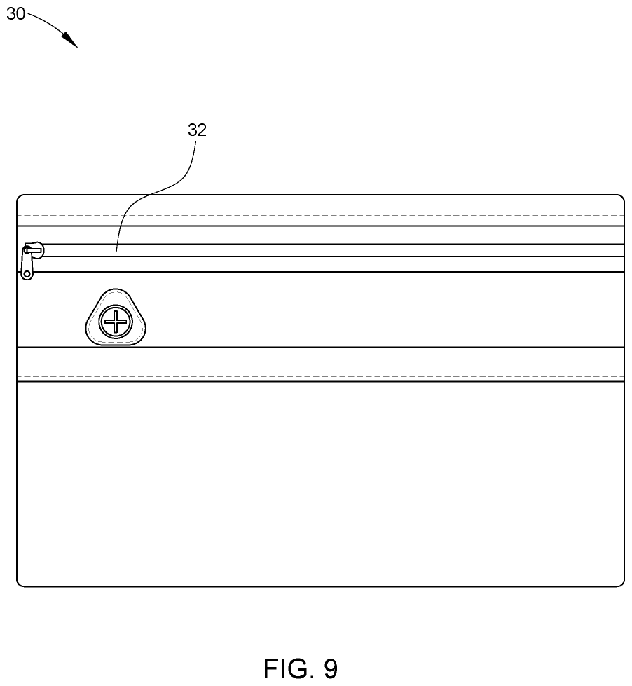

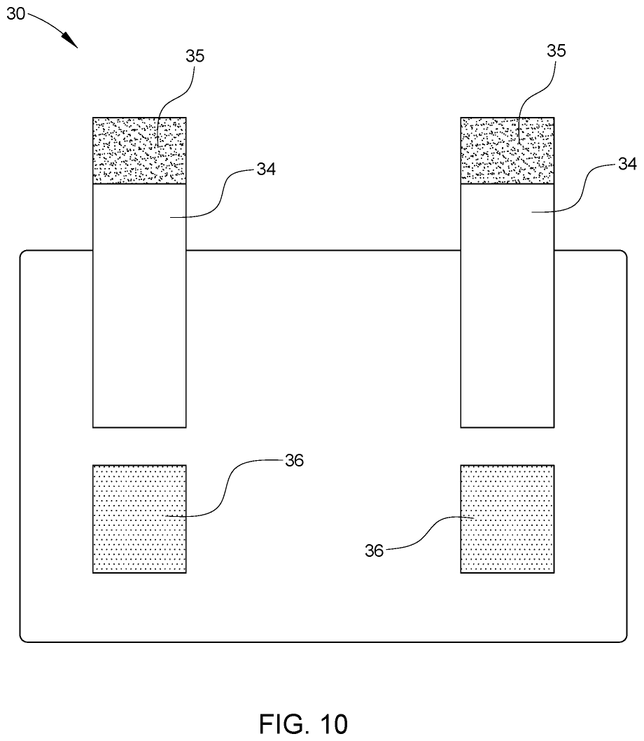

As illustrated in FIG. 9, the exercise system of the present invention can further include pouch 30 with zipper 32 for receiving and storing the folded exercise system of the present invention, or other items such as a wallet, credit cards, and the like. The pouch can be used as a stand-alone carrier or can be attached to the belt 16 by stripes 34 having Velcro material 35 and 36 as shown in FIG. 10, for example. It will be understood by a person skilled in the art that the pouch 30 can be attached to the belt 16 using other suitable attaching means such as snap buttons, magnetic closures, buckles, D-rings or carabiners, zippers, clips, or adhesive attachments.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

Although the invention is described herein with reference to specific embodiments, various modifications and changes can be made without departing from the scope of the present invention as set forth in the claims below. Accordingly, the specification and figures are to be regarded in an illustrative rather than a restrictive sense, and all such modifications are intended to be included within the scope of the present invention. Any benefits, advantages, or solutions to problems that are described herein with regard to specific embodiments are not intended to be construed as a critical, required, or essential feature or element of any or all the claims.

Unless stated otherwise, terms such as “first” and “second” are used to arbitrarily distinguish between the elements such terms describe. Thus, these terms are not necessarily intended to indicate temporal or other prioritization of such elements.

The foregoing detailed description is merely exemplary in nature and is not intended to limit the invention or application and uses of the invention. Furthermore, there is no intention to be bound by any expressed or implied theory presented in the preceding technical field, background, brief summary, or the following detailed description.

What is claimed is:

1. An exercise system for integrating physical activity with mental engagement to promote both physical exercise and mental involvement, reducing stress, anxiety, and enhancing cognitive performance as well as improving overall well-being, said system comprising:
a harness adapted to wrap around and hold a ball coupled to a length adjustable belt by a length adjustable band.

2. The exercise system of claim 1, wherein the corners of the harness each terminating in a stripe for wrapping around and holding the ball.

3. The exercise system of claim 2, wherein the harness further comprising two crossed extension stripes attached to one of the stripes of the harness.

4. The exercise system of claim 1, wherein the harness is a net adapted to receive and hold the ball.

5. The exercise system of claim 1, wherein the harness is comprised of a plurality of longitudinally oriented bands supportingly attached to a plurality of laterally oriented bands at cross points, wherein the bands are substantially thin smooth surfaced bands to minimize radial protrusion from the surface of the ball.

6. The exercise system of claim 1, wherein the band is permanently attached to the belt and the harness.

7. The exercise system of claim 1, wherein the band is releasably attached to the belt and the harness by virtue of Velcro strips, D-rings, carabiners, hook and loops, or combination thereof.

8. The exercise system of claim 1, wherein the band is elastic.

9. The exercise system of claim 1, wherein the belt is elastic.

10. The exercise system of claim 1, further comprising a pouch for receiving and storing one or more items.

11. The exercise system of claim 10, wherein the pouch further comprising a zipper.

12. The exercise system of claim 10, wherein the back side of the pouch further comprising attachment means for releasably attaching the pouch to the belt.

13. The exercise system of claim 12, wherein the attachment means are stripes with Velcro material, snap buttons, magnetic closures, buckles, D-rings, carabiners, zippers, clips, or combination thereof.

14. The exercise system of claim 1, wherein the maximum length of the band is 11 inches (27.94 cm).

15. The method of using an exercise system for integrating physical activity with mental engagement to promote both physical exercise and mental involvement, reducing stress, anxiety, and enhancing cognitive performance as well as improving overall well-being, the method comprising:
providing the exercise system comprising a harness adapted to wrap around and hold a ball coupled to a length adjustable belt by a length adjustable band;
encircling the harness around the ball;
securing the belt around the user's waist;
adjusting the length of the band as to position the ball at or slightly above the knee level; and
kicking the ball using either knee.

16. The method of claim 15, wherein the maximum adjustable length of the belt is 11 inches (27.94 cm).
